(19) United States
(12) Patent Application Publication
(10) Pub. No.: US 2007/0250148 A1
(43) Pub. Date: Oct. 25, 2007

(54) Systems, apparatus and methods related to helical, non-helical or removable stents with rectilinear ends

(76) Inventors: Kenneth E. Perry JR., Bainbridge Island, WA (US); Paul E. Labossiere, Bainbridge Island, WA (US); Gifford Pinchot, Bainbridge Island, WA (US)

Correspondence Address: GRAYBEAL, JACKSON, HALEY LLP, 155 - 108TH AVENUE NE, SUITE 350, BELLEVUE, WA, 98004-5973, US

(21) Appl. No.: 11/729,309

(22) Filed: Mar. 27, 2007

(30) Foreign Application Priority Data
Sep. 26, 2005 (US) PCT/US05/34482

(51) Int. Cl. A61F 2/84; A61F 2/88; A61F 2/90

(52) U.S. Cl. 623/1.11; 623/1.16; 623/1.22

(57) ABSTRACT

An intraluminal stent comprising a helical arrangement of elements defined by a successive series of substantially straight struts connected by apex sections alternately pointing in the opposite directions, wherein at least one apex section comprises two struts attached thereto with a length ratio about 1:2.

### CROSS-REFERENCE TO RELATED APPLICATIONS

The present application claims priority from PCT Application No. PCT/US2005/034482, filed Sep. 26, 2005 which claims priority from U.S. provisional patent application No. 60/613,677, filed Sep. 27, 2004, and from U.S. provisional patent application No. 60/634,683, filed Dec. 8, 2004, which are incorporated herein by reference in their entirety and for all their teachings and disclosures.

### BACKGROUND

Generally speaking, a stent is an expandable tube, usually made of wire mesh, that is inserted into a hollow structure of the body such as a blood vessel to keep it open. Thus, one of the typical ways of treating disease such as clogging of the arteries (atherosclerosis), stenoses, strictures, thrombosis, or aneurysms is to place a stent into the affected vessel. Among other advantages, stents reduce the chance the vessel(s) will collapse, increase cross sectional area (and thereby increase the amount of blood that can flow), and reinforce the vessel walls. Many stents have been developed, and the prior art includes a wide variety of types and methods for their manufacture.

Typically, the structural form for stents, stent grafts, heart valve frames and the like is a circumferential architecture, where hoops are arranged sequentially along a longitudinal axis. This provides a discrete series of supporting hoops for the vessel receiving the stent. An exemplary stent is depicted in U.S. Pat. No. 6,342,067. In another example, a helical stent has helical windings connected by bridges where the bridges are not in a circumferential plane, which can provide improved flexibility and kink resistance. Traditional helical stents typically comprise strut ratios of about 1:1 to about 1:1.1 and have either non-squared ends or a “transition zone,” for example between the helical portion of the stent and a hoop-shaped end of the stent, which can make it difficult to achieve uniform performance properties over the length of the stent. Exemplary helical stents are depicted in WO 01/89421 A2, U.S. Pat. No. 6,042,597, USPA 20040143318, USPA 20040054398, W00234163(A2), USPA 20040106983, USPA 20040093076, and USPA 20040044401 (these and all other references herein are incorporated herein by reference in their entirety and for all their teachings and disclosures, regardless of where the references may appear in this application).

Thus, there has gone unmet a need for improved stents having improved combinations and/or consistency of characteristics along the full length of the stent, and/or to resist kinking. The present devices, systems and methods provide one or more of these or other advantages.

### SUMMARY

In one aspect, the present discussion is directed to helical stents with excellent performance properties including at least one of flexibility, vessel conformity, adaptation, blood flow dynamics, durability, substantially uniform vessel scaffolding with kink resistance, and large open holes if desired. The stents can be partially or fully retracted back into the delivery catheter during deployment or otherwise as desired for precise positioning. The stents can be compressed to catheter dimensions prior to introduction to achieve a low delivery profile and can self-expand to its fully intended diameter within the lumen of the target vessel.

In one embodiment, the stent comprises a plurality of nesting helical or non-helical windings (which may be generated by one or more individual elements). In the case of the helical windings, the windings are generally aligned with the longitudinal axis. (Unless expressly stated otherwise or clear from the context, all embodiments, aspects, features, etc., can be mixed and matched, combined and permuted in any desired manner.) Each winding comprises adjacent strut pairs (defined as two struts connected at a single apex) comprising a short strut and a long strut that have integral length ratio relative to each other, which means that the ratio can be expressed in whole numbers. For example, the short strut may be approximately 1 unit in length and the long strut can be approximately 2 units in length, to give a 1:2 ratio. Ratios such as 1:3, 1:4, etc., are also possible. This configuration progresses the winding along the helical axis. In some embodiments, the short struts of various strut pairs can be substantially all of the same length, or they can vary such that corresponding members of short struts are one length while the opposing members of the paired short struts are another length. Thus, if desired, the lengths of the short struts can be configured to additively equal substantially the length of the long struts (e.g., one short strut of 40% the length of the long strut and one short strut of 60% the length of the long strut), or to be more or less than the length of the long struts such that the combination of such lengths themselves provide a helical aspect to the pattern of the windings (e.g., one short strut of 40% the length of the long strut and one short strut of 80% the length of the long strut).

The stent can be provided with or without a graft or covering, and in certain embodiments the graft can supplant all or substantially all bridges in the stent. The stent can also be coated with an agent, such as heparin or rapamycin, to inhibit stenosis or restenosis of the vessel, or a biological or biomemetic coatings that can be for inhibiting stenosis or restenosis or other reasons. Examples of such coatings are discussed in U.S. Pat. Nos. 5,288,711; 5,516,781; 5,563,146 and 5,646,160.

In other embodiments, the stents can comprise one or more windings with substantially no bridges. If desired, the stent can have no bridges, bridges only at the ends to eliminate “loose ends” of the windings (if the windings have loose ends), or only a very few bridges throughout the stent, enough to provide adequate longitudinal and axial support to avoid kinking under physiological stress levels yet still provide desired longitudinal and axial flexibility and expandability, typically while also providing predictable, known minimum longitudinal and axial dimensions.

If desired, a plurality of stents can be provided as a stent system or a kit (the stents discussed elsewhere herein can be also provided in kits, if desired), in which the stents provide a virtually limitless variety of bridging options, short strut v. long strut iteration ratios, short strut v. long strut length ratios (and short v. short and long v. long strut ratios), materials, or other features affecting flexibility, expandability (axial and/or longitudinal), winding diameter, twistability, minimum/maximum diameter and/or length, etc. If desired, a set of stents providing a predetermined variety of such options can be provided. If further desired (or instead of, in some embodiments), “custom-made” stents having other specifically desired properties can be individually or collectively ordered and created to meet specific needs of a physician or other health care provider.

In some embodiments, computer implemented programs can comprise information such as that provided herein and then automatically configure the stent bridging, strut ratios, etc., to provide stents of desired flexibility, expandability (axial and/or longitudinal), winding diameter, twistability, minimum/maximum diameter and/or length, etc. In other words, instead of a practitioner instructing a computer or other manufacturing device to make a stent having a certain desired windings configuration, ratios, etc., the practitioner can ask the computer for a stent having certain desired flexibility, expandability (axial and/or longitudinal), winding diameter, twistability, minimum/maximum diameter and/or length, etc., characteristics, and then the computer can determine a suitable physical configuration for the stent.

Certain benefits of certain embodiments here arise from the helical architecture of the stent. In certain embodiments desirable benefits can also, or instead, arise from an architecture that does not include a transition zone (such as in WO 01/89421 A2, U.S. Pat. No. 6,042,597, USPA 20040044401 and USPA 20040143318) and therefore certain desirable properties of the stent are uniform and continuous from one end of the stent to the other.

These and other aspects, features and embodiments are set forth within this application, including the following Detailed Description and attached drawings. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE FIGURES

FIG. 1A is a schematic view depicting an exemplary, flattened stent pattern as discussed herein. A single helical winding is darkened for highlighting.

FIG. 1B is a schematic view of the same stent pattern in FIG. 1A in an expanded state.

FIG. 2 is a schematic view depicting further exemplary, flattened stent patterns and illustrating a variety of bridge patterns that can be used to get slightly different combinations of properties.

FIG. 3 is a schematic view depicting another exemplary, flattened stent pattern as discussed herein.

FIG. 4 is a schematic view depicting another exemplary, flattened stent pattern comprising multiple short strut pairs located between approximate 1:2 ratio strut pairs.

FIG. 5 is a perspective view of an exemplary, prior art, proximal end latching connector that is one example of an end latching connector that can be used with the stents herein.

FIG. 6 is a schematic view depicting a further exemplary, flattened stent pattern as discussed herein that depicts a further general strut ratio and layout pattern.

FIG. 7 is a schematic view depicting still another exemplary, flattened stent pattern as discussed herein that depicts a further general strut ratio and layout pattern.

FIG. 8 is a schematic view depicting still an exemplary, flattened stent pattern as discussed herein that comprises a coupled torsional/diametric response (i.e., a variable diameter response upon twisting of the stent).

FIG. 9 is a schematic view depicting still another exemplary, flattened stent pattern as discussed herein that comprises a coupled torsional/diametric response.

FIG. 10 illustrates an example of how a stent can be removed from an implantation site where the distal end of the stent has become anchored in the lumen by twisting the proximal end of the stent to cause the stent diameter to decrease.

FIG. 11 illustrates another example of stent removal from an implantation site where the distal end of the stent has not become anchored in the lumen by use of an inflatable balloon that holds the distal end of the stent to the lumen wall.

FIG. 12 illustrates a further example of stent removal where one or more rows of circumferential hoops that can be shape set at different unchanging diameters provide a press-fit between the stent and the lumen wall.

FIGS. 13A-C illustrate three examples of a strut profile configured to encourage localized twisting or bending, etc.

FIG. 14 depicts an exemplary stent repeat pattern that includes struts that preferentially deform in response to a torsion load and strut pairs that preferentially deform in response to a bending load.

FIG. 15 depicts an exemplary stent repeat pattern that includes a “wishbone” configuration between adjacent long struts in the end winding and multiple short struts between long struts in the interior windings.

FIG. 16 depicts an exemplary stent repeat pattern that includes multiple short struts between long struts in the windings.

### DISCUSSION

In some aspects, the apparatus, systems, methods, etc., discussed herein comprise stents with very good performance properties including one or more of excellent flexibility, vessel conformity, adaptation, blood flow dynamics and durability. Certain benefits arise from the helical architecture of the stent in some embodiments. In some embodiments, the stents can have substantially squared or rectilinear ends and uniform architectural properties from one end of the stent to the other and/or can be configured with a distribution of bridges and integral ratio-strut pairs that provide desired articulation of the stent.

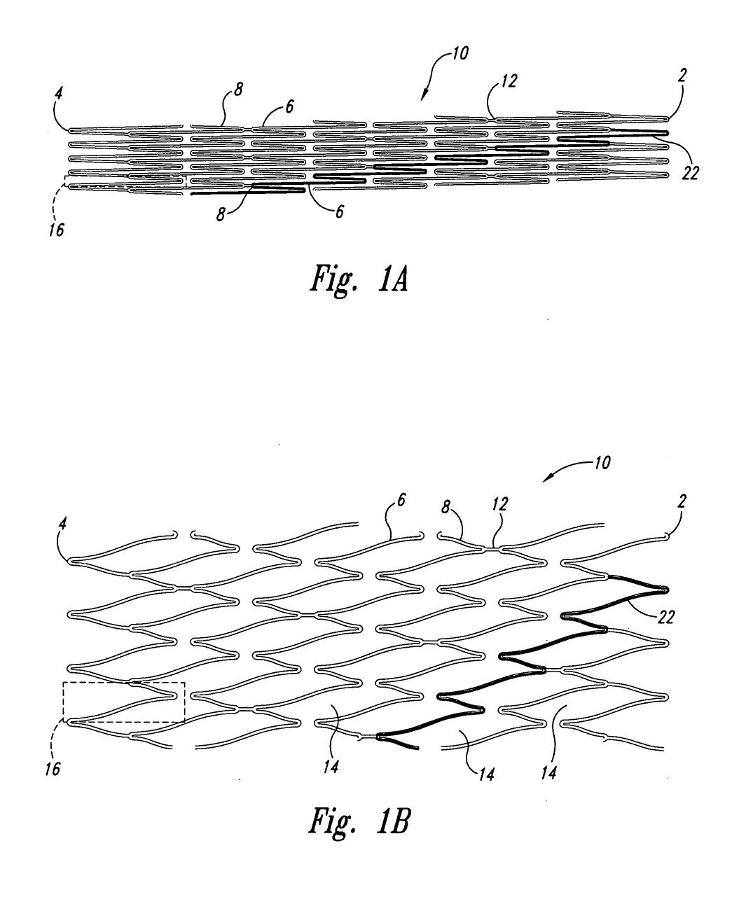

Turning to a discussion of the Figures, FIG. 1A depicts a schematic view of an exemplary, flattened stent pattern comprising helical nested windings. In FIG. 1A, a stent 10 has a distal end 2 and a proximal end 4 and several helical windings 22 made of wire, plastic or other suitable material. The helical windings 22 in this embodiment comprise a plurality of adjacent strut pairs 16 having a long strut 6 and a short strut 8 having an integral 1:2 ratio. Selected apexes of the strut pairs are joined by bridges 12. A single helical winding 22 is darkened to highlight the path of such windings. FIG. 1B is a schematic view of the same stent pattern in FIG. 1A in an expanded state. In the expanded state, a plurality of openings 14 are created. Such openings 14 can be used to access the interior of the stent 10 from the outside, or to access the lumen or other target from the inside of the lumen and the stent 10.

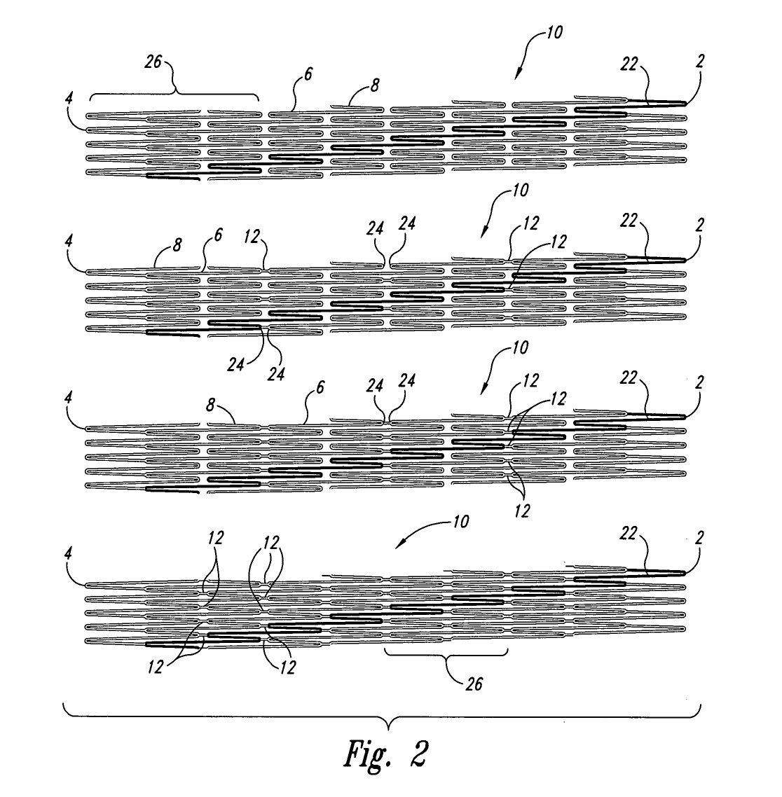

FIG. 2 provides a schematic view showing further exemplary patterns of bridges 12 for stents 10 that can be used to obtain different combinations of properties in the stents 10, such as different torsional, bending, elasticity, etc., properties. The first embodiment has no bridges and is thus the most flexible and open. The second embodiment has balanced intermittent bridges 22 between opposing apexes 24, and thus has intermediate properties and is more symmetric than certain other embodiments noted herein. The third embodiment has an alternating configuration of bridges where all opposing apexes 24 of a given pair of windings 22 are bridged but opposing apexes 24 of the intermediate windings 22 are not bridged. Such an embodiment has intermediate properties that are more rigid than the second embodiment of FIG. 2. The fourth embodiment has bridges 12 at substantially every juncture between opposing apexes 24 resulting in the most rigid structure of the embodiments in FIG. 2. As can be seen, in these embodiments opposing strut pairs 26 are bridged at the respective peaks of the strut pairs. In some other embodiments, the struts can be bridged at other locations, for example directly from strut to strut such as is shown in USPA 20040143318.

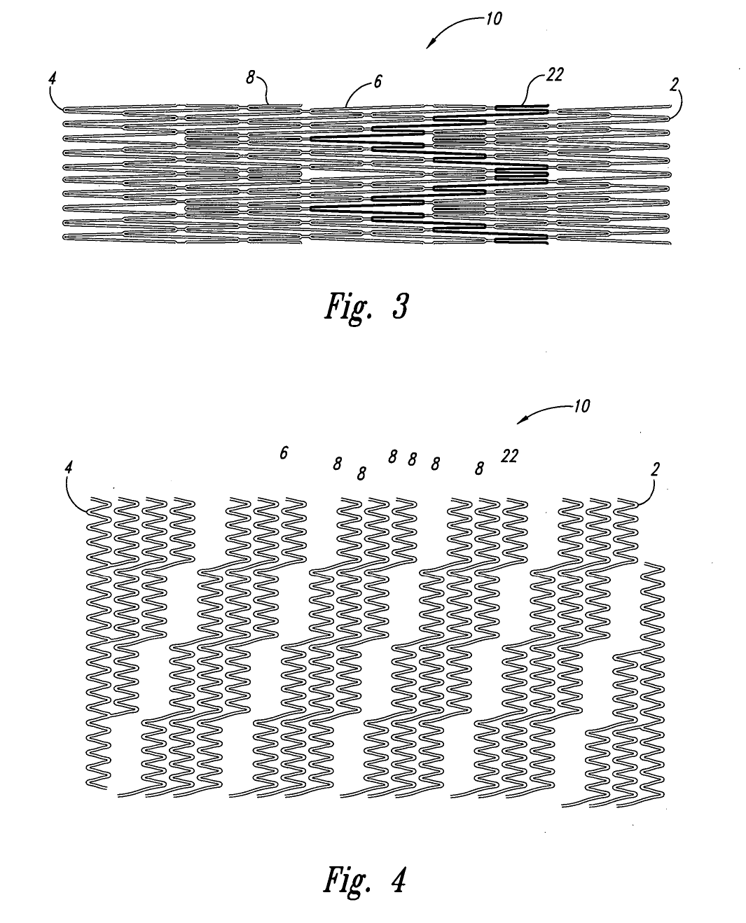

FIG. 3 is a schematic view showing another exemplary pattern for a winding 22 of another stent 10. In this embodiment the pattern is repeated according to two-fold symmetry such that it loops back and forth through the stent. Other multi-fold symmetry is also possible, such as three-fold or four-fold symmetry.

FIG. 4 is a schematic view showing a further exemplary pattern for helical windings 22 for a stent 10. In this embodiment the helical windings 22 comprise several short struts 8 for each long strut 6. Such configuration provides still further possible combinations of flexibility, vessel conformity, adaptation, blood flow dynamics and durability, torsional, bending, elasticity, etc.

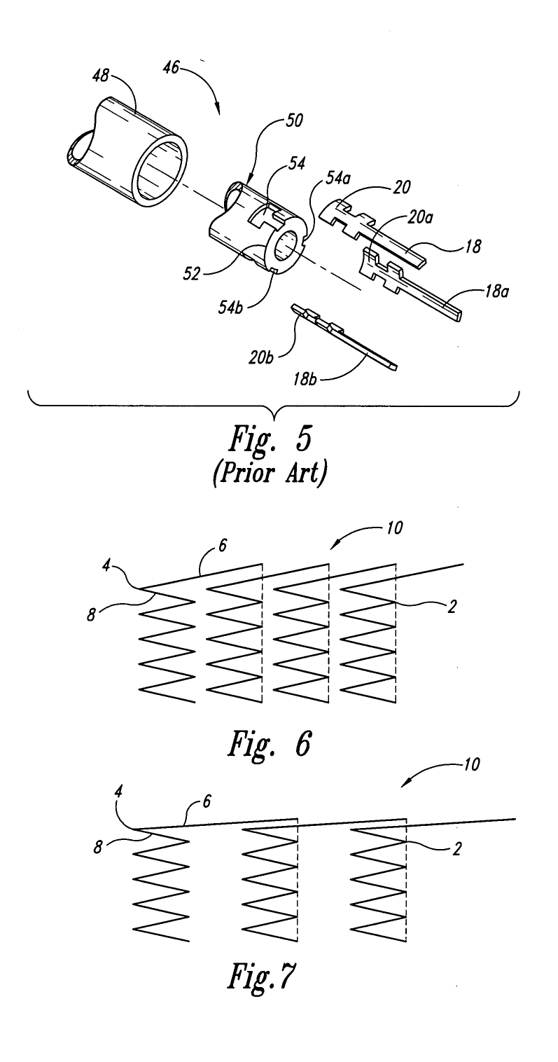

FIG. 5 illustrates an exemplary deployment system 46 that can be used with a stent 10 herein. The deployment system 46 includes an outer sheath 48 which is essentially an elongated tubular member, similar to ordinary well known guiding catheters . The deployment system 46 also includes an inner shaft 50 located coaxially within the outer sheath 48 prior to deployment. The inner shaft 50 has an end 52. The distal end 52 of the shaft 50 has three grooves 54, 54a, and 54b disposed thereon. When the deployment system 46 is not fully deployed, the stent device 10 is located within the outer sheath 48. The T-shaped or I-shaped attachment flanges 20, 20a, and 20b on the proximal legs 18, 18a, and 18b are set within the grooves 54, 54a, and 54b of the inner shaft 50, thereby releasably attaching the stent device 10 to the inner shaft 50. This deployment system also discussed in U.S. Pat. No. 6,267,783.

FIGS. 6 and 7 provide schematic views showing helical windings 22 for a stent 10 comprising several short struts 8 for each long strut 6, and wherein the sinusoidal waves of the windings 22 are angled in a Z fashion. In FIG. 7, short struts 8 and long struts 6 have an integral 1:3 length ratio. Again, such configurations provide still other combinations of flexibility, vessel conformity, adaptation, blood flow dynamics and durability, torsional, bending, elasticity, etc.

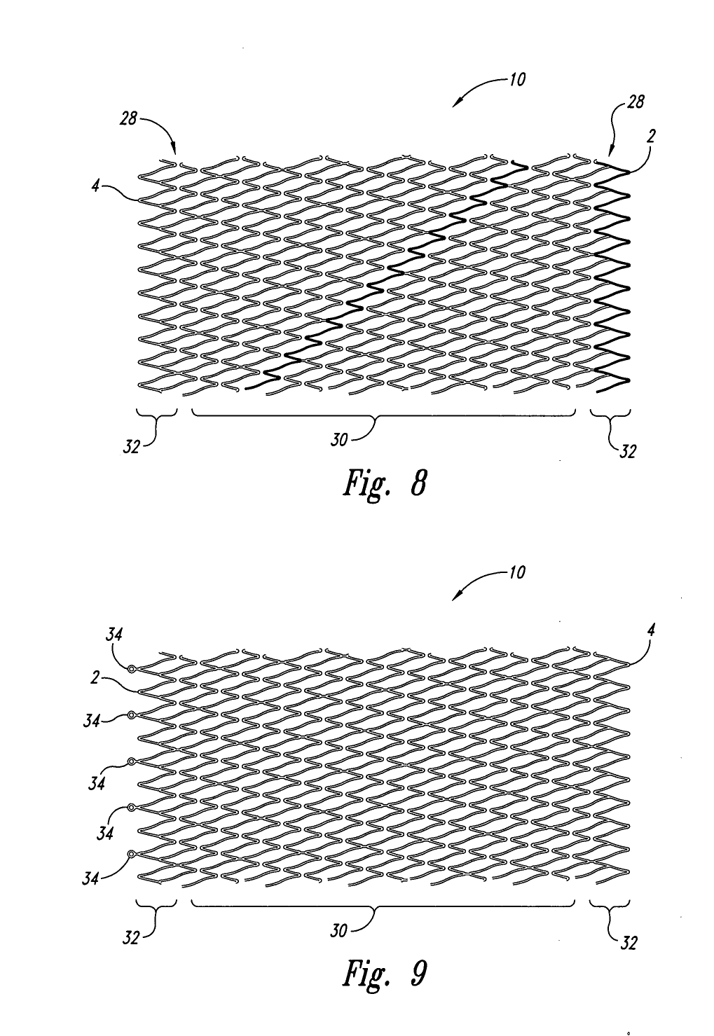

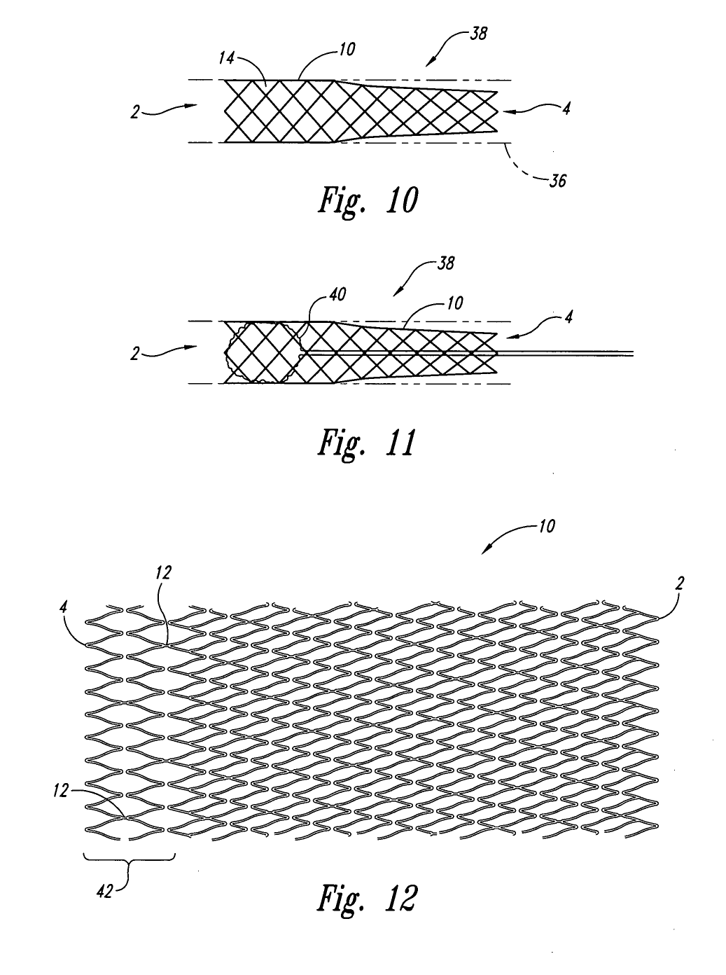

As depicted in FIG. 8, in some embodiments the stents 10 can have different properties at different locations. For example, changeover zones 28 can be located between variable diameter sections 30 and non-variable diameter sections 32 of the stent 10 such that one portion of the stent comprises variable diameter architecture while another part of the stent does not. In some embodiments a distal end 2 of the stent can comprise variable diameter architecture while the proximal end 4 does not, or vice-versa. As another example, each of the distal end 2 and proximal end 4 can comprise variable diameter architecture while the middle does not, or vice-versa as in the embodiment shown in FIG. 10 (i.e., each of the distal and proximal ends does not comprise variable diameter architecture while the middle does). Other combinations and configurations of variable/non-variable diameter architecture are also possible as desired.

FIG. 9 illustrates grips 34 on the proximal end 4 of the stent 10 that can be included in the stent 10 to facilitate grasping the stent (before, during or after implanting) prior to twisting. Grips 34 as shown are loops, but other grips such as hooks can also be used. Alternatively, the bare ends of the stent 10, such as those shown at the proximal end 4 may be grasped without hooks or other specialized grips. Both twisting and/or pulling loads can be applied to the end of the stent 10 to insert, reposition or fully remove the stent 10 from an implanted site. If desired, the load(s) can also be applied inside the ends of the stent. The stent can be pulled directly out of the body (for example from accessible implantation sites such as the urethra, esophagus, etc.) and/or the stent can be pulled into a catheter that may or may not have a flared distal tip to accomplish further diametric compression to an even smaller diameter.

FIG. 10 illustrates an example of how the stent 10 can be removed from an implantation site 38 where the distal end 2 of the stent has become anchored in the lumen 36, for example due to tissue in-growth wherein tissue grows through openings 14 of the stent 10. As can be seen, twisting the proximal end 4 of the stent 10 causes the stent diameter to decrease, thereby releasing the stent from the lumen wall.

FIG. 11 provides one approach to stent removal where tissue in-growth does not provide sufficient anchoring to counteract the twisting force applied to the proximal end 4 of the stent 10. In this case, the distal end 2 can be anchored using other approaches such as an inflatable balloon 40.

FIG. 12 depicts another method to provide anchoring of the distal end 2 of the stent 10. In this embodiment, the distal end 2 has one or more rows of circumferential hoops 42 that can be shape-set at different unchanging diameters to provide a press-fit between the stent and the lumen wall.

In other approaches, the stent can comprise other specific features or variations to increase (or decrease, if desired) the anchoring force at the distal end of the stent. For example, the stent can have multiple segments where each segment has a different twist/expansion ratio and in some embodiments both an increase and a decrease may be obtained for different segments given the same twist by reversing the helical pitch direction.

The stent can include one or more struts or patterns of struts where the thickness, width, strength and/or length of the struts are varied in relation to neighboring struts or patterns of struts to create a predominantly torsional mode of deformation in some struts under general or specific loading conditions. For example, some struts can have reduced strut widths in the middle of their length and larger widths near their apexes such that under certain stent loading conditions these struts will tend to deform by twisting about the reduced section rather than deforming over their length.

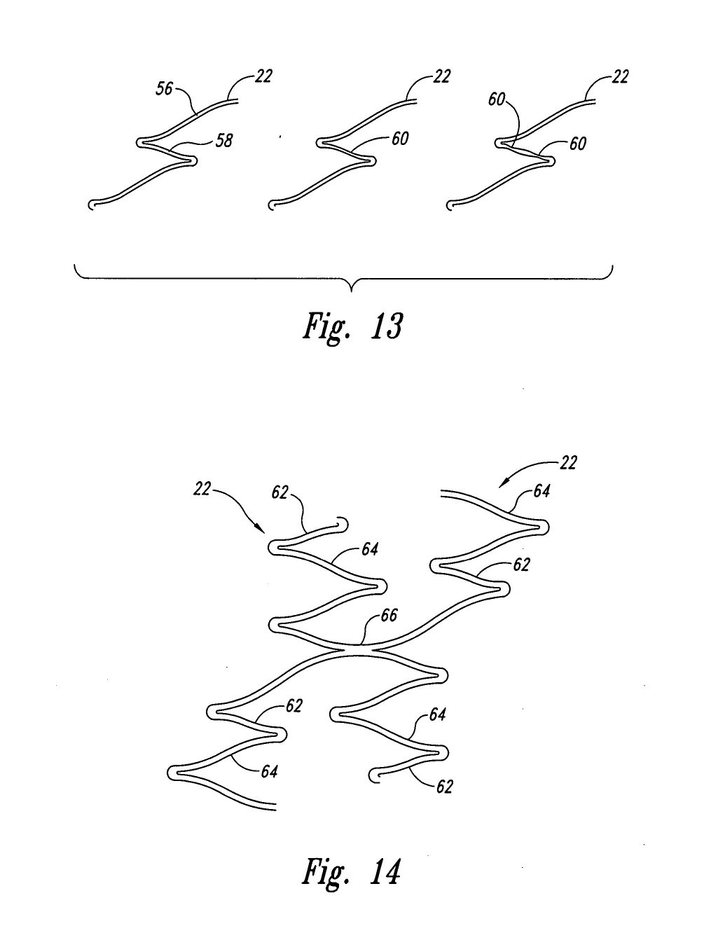

FIGS. 13A-C illustrate three examples of how a strut profile may be configured to encourage localized twisting or bending, etc. FIG. 13A shows an adjacent strut pair 16 without any narrowings to provide such localized twisting or bending, but the winding 22 is comprised of multiple materials, a softer, more malleable material in malleable zone 56 and a harder, more rigid material in rigid zone 58. FIGS. 13B and 13C show adjacent strut pairs 16 with narrowings 60 to provide the localized twisting or bending.

This localized property effect may be further enhanced by providing patterns of struts within the stent that are favorably disposed to a given loading condition (e.g., twisting one end). Alternatively, some struts may be configured to respond more favorably to one particular loading condition while other struts are configured to respond more favorably to a different loading condition (e.g., uniform diametrical compression). Combinations of various deformation configurations and characteristics can be included to improve catheter crimping, deployment, positioning, extraction, long term response to physiological deformations, etc.

FIG. 14 is an exemplary stent repeat pattern that includes struts that preferentially deform in torsion (torsion-deformable zone 62) and strut pairs that preferentially deform in bending (bending-deformable zone 64). One of many variations of suitable bridges (link between adjacent regions) is also shown in the center of FIG. 14 in the form of a direct bridge 66.

The bridges can also be tailored to respond in specific ways to various stent loading conditions. In some cases, the bridges can be tailored to maximize a torsional/diametric response. In other embodiments the bridges may be configured to respond in concert with other patterns of struts to provide a multi-stage response to a specified loading condition or improved fatigue properties.

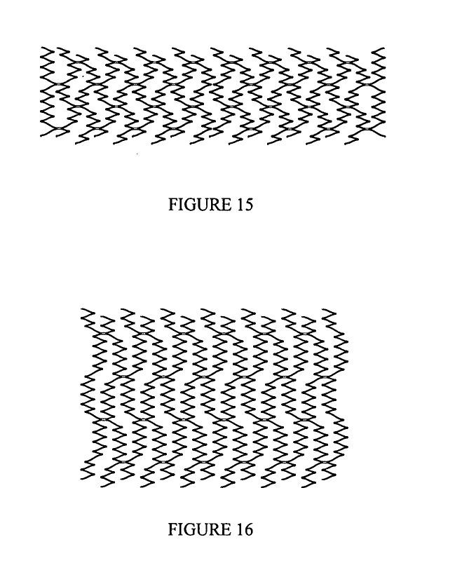

FIG. 15 depicts a stent 10 having an exemplary stent repeat pattern that includes a “wishbone” configuration 68 between adjacent long struts in the non-helical, square-ended end winding 70 and multiple short struts 8 between long struts 6 in the non-helical interior windings 72.

FIG. 16 depicts a stent 10 having a repeat pattern that includes multiple short struts 8 between long struts 6 in the non-helical windings, and that lacks a square-ended end winding.

Turning to a further general discussion of the stents, etc., herein, the stents can be used to integrate the benefits of helical structures with typical circumferential structural stent elements such as hoops since the underlying helical structure has a reciprocal rectilinear repeat pattern. For example in the case of a frame for a heart valve, sections of nested helical windings can be located between circumferential hoop elements providing enhanced flexibility and articulation between the hoops with a range of desired bending, torsional, axial and radial support properties depending for example on the stent material, the diameter of the struts, the configuration, composition and/or spacing of the bridges, the precise ratio of the strut ratio pairs, the combination of differing materials, etc. Multi-fold embodiments can further provide tissue attachment and other reinforcement structures.

In certain embodiments, the current stents have nesting elements joined by connectors or bridges between linked apexes such that the entire structure can be compressed to a smaller diameter prior to delivery to the lumen of the intended vessel. The apex connections can be horizontal, vertical, at an angle, all the same or not and thus different variations can lead to stents with desired properties.

Furthermore, the stents can have an open cell structure, which can be advantageous for applications where it is desired to pass catheters or other minimally invasive tools through the cells of the stents. The nested about 1:2 strut ratio pairs (and other integral ratio pairs) provide excellent access for such tools while maintaining adequate pressure and coverage to the vessel. Other strut pair ratios can be provided in other embodiments, for example from about 1:1.5 or greater (for example in non-integral ratios, if desired the stent may comprise helical windings and helical, non-rectilinear repeat pattern).

In some embodiments, the stents can be partially or fully retracted or recaptured during catheter deployment. This feature, in some embodiments, arises from the substantially longitudinal orientation of the nested helical windings, which present a series of continuous and uniform elements at the distal end of the deployment catheter. In some embodiments it may be desired to slightly bias the proximal apex of each (or most) short strut in an asymmetric pair slightly inward. The furling/unfurling elements of the stent are substantially longitudinal and permit bidirectional motion of a restraining sheath.

U.S. Pat. No. 6,673,106 discusses a stent that is retractable and includes a proximal end latching connector reproduced here as FIG. 5 and discussed above. Such connectors are exemplary of connectors that can be used with the stents herein to achieve retractability during deployment. The current stents achieve retractability through an improved strategy for aligning the stent elements essentially along the longitudinal axis thus providing a retractably smooth condition at the distal tip of the catheter during deployment. Other desired structures can also be combined with the structures and methods discussed herein.

The stent can be provided with or without a graft or covering, and in certain embodiments the graft can supplant all or substantially all bridges in the stent. The stent can also be coated with an agent, such as heparin or rapamycin, to prevent stenosis or restenosis of the vessel. Examples of such coatings are discussed in U.S. Pat. Nos. 5,288,711; 5,516,781; 5,563,146 and 5,646,160.

In certain embodiments the stents can also be effectively miniaturized, for example because the strut pairs can be configured to overlap in a non- or low-obstructive fashion. Thus, more and/or wider struts can be used for a given delivery catheter resulting in greater radial force available for the intended vessel.

In further aspects, the stents herein are configured to be capable of changing diameter upon twisting about the longitudinal axis. Twisting can produce either an increase or a decrease in diameter depending on the direction of the twist and the specific architecture of the stent. This feature can be utilized, for example, in an implantable stent device where a reduction in diameter can facilitate insertion, repositioning or extraction of the stent or where an increase in diameter can be used to enlarge a healthy or diseased lumen, or to assist in maintaining the stent in its desired position. Certain aspects of the expandable and retractable configurations can also be incorporated into non-implanted devices and other applications as an aspect of minimally invasive surgical tools capable of positioning, recapturing, anchoring, expanding and/or otherwise manipulating devices and performing treatment.

Depending on the selected configuration of the architecture and configuration of the stent, all or only a fraction of the available diametric change (either an increase or decrease) may be controlled by twisting, so that increasing or decreasing the amount of twist correspondingly increases or decreases the amount of diametric change. Additional diametric change can be accomplished by other mechanisms such as uniform radial compression, either before, during or after a twist has been applied.

The stent can be a bare stent or it can also incorporate a graft material. The stent can be made from a superelastic or other metallic, plastic or otherwise suitable material and also can be made from a conventional polymer or biodegradable polymer. As a bare stent or a stent graft, the open space between struts can allow tissue and in-growth which can be helpful for anchoring the implant while still providing for removability. Alternatively, a different configuration for a stent graft may be constructed to minimize tissue in-growth.

The twist can be temporary (while the twist is applied) or “locked in” through friction or by providing a static latch or other structure to restrain the twist. The restraint method could include barbs for anchoring directly in tissue or other methods for securing specific locations of the stent such as sutures.

The stent can be configured to exhibit an outward force to support and hold open a lumen or it can be configured to provide an inward force capable of shrinking, squeezing, sealing, etc.

The configurations incorporated in the stents discussed herein can also be used in other related purposes such as in a frame for mounting a heart valve. Other exemplary uses include treatment of benign prostatic hyperplasia, removable stents for the bronchi, esophagus and airway as well as minimally invasive procedures comprising temporary or permanent vessel dilation and support.

The various features provided herein also comprise methods of making and of using the stents discussed herein, including methods that comprise multiple embodiments, combinations and permutations of the various features of the stents discussed herein.

Various aspects of the stents herein that can be varied include without limitation:
short/long struts of the stents can have different widths and can be tapered.
bridge configurations (numbers, placement and patterns) can be modified to get different overall flexural properties.
Either or both short and long struts can be removed from the stent's pattern to further change deformation and/or articulation properties.
the stents can have multi-fold symmetry for applications involving tissue mounting, connections to other components, bifurcations, etc.
the stents can be made of any desired suitable engineering material for implantation, such as metal, polymer (e.g., composite, drug eluting, bioerodable).
the stents can comprise multiple runs of short struts between long struts.

Thus, in some embodiments the present discussion can be directed to helical stents sized and configured for insertion into a lumen of a vessel of a patient, comprise at least one or more nesting helical winding having at least one adjacent strut pair having an integral length ratio of at least about 1:2, 1:3, 1:4 or more.

The helical stents can also comprise multiple nesting helical windings having uniform strut lengths and comprise at least one pair of adjacent struts with a length ratio of about 1:2, and/or can be easily flexible, expandable rectilinear helical stent comprising substantially squared ends and substantially uniform architectural properties throughout the stent, generally lacking any transition zone.

The stent can be configured to comprise substantially only the nesting helical windings, and/or can be further configured such that a diameter of the stent at least one of controllably expands or controllably contracts upon twisting of the stent. The stent can further comprise grips such as loops, hooks, extensions, flanges, etc., configured to be grasped for the twisting.

The stent can have multiple segments with at least one variable diameter segment configured such that a diameter of the segment at least one of controllably expands or controllably contracts upon twisting of the stent, and at least one non-variable diameter segment that substantially does not expand or contract upon twisting of the stent. The stents can further comprise at least one second variable diameter segment, for example wherein the diameter of the second variable segment upon twisting can be different from the diameter of the first variable segment upon the twisting.

The stent can comprise at least one non-variable segment disposed between at least two variable segments, and/or at least one variable segment disposed between at least two non-variable segments. The segments can have same or different twist/expansion ratio(s). The segments can be configured such that a diameter of at least one variable segment increases and a diameter of at least one other variable segment decreases by a single twist of the stent.

The stents can also comprise at least one lock configured to maintain the stent at a desired diameter after the stent has been twisted.

The stents can be configured for implantation into any desired biological space, such as a vascular or neural cavity. The stent can be cut from small diameter tubing and can be expandable to a final diameter, can be cut from cut from tubing having a diameter that can be substantially the same as the final diameter of the stent after implantation, and can be constructed of any desired material such as wire, ribbon, thin sheet, implantable metal, stainless steel, Nitinol, cobalt, chrome, superelastic material or polymer, or any other material with adequate mechanical strength.

The present application also includes methods comprising making and/or using a stent as discussed herein, for example by implanting the stent into a lumen of a vessel of a patient.

The scope of the present systems and methods, etc., includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims.

What is claimed is:

1. A stent sized and configured for insertion into a lumen of a vessel of a patient, comprising at least one nesting winding having at least one adjacent strut pair having an integral length ratio of at least about 1:2.

2. A stent configured for insertion into the lumen of a vessel of a patient, comprising multiple nesting windings having uniform strut lengths and comprising at least one pair of adjacent struts with a length ratio of about 1:2.

3. An easily flexible, expandable stent comprising a rectilinear pattern of helical segments comprising substantially squared ends and substantially uniform architectural properties throughout the stent.

4. The stent of claim 2 wherein the stent lacks any transition zone.

5. The stent of claim 3 wherein the stent lacks any transition zone.

6. The stent of claim 2 wherein the stent is configured to comprise substantially only the nesting windings.

7. A stent sized and configured for insertion into a lumen of a vessel of a patient, the stent further configured such that a diameter of the stent at least one of controllably expands or controllably contracts upon twisting of the stent.

8. The stent of claim 7 wherein the stent further comprises grips configured to be grasped for the twisting.

9. The stent of claim 7 wherein the stent comprises multiple segments, the multiple segments comprising at least one variable diameter segment configured such that a diameter of the segment at least one of controllably expands or controllably contracts upon twisting of the stent, and at least one non-variable diameter segment that substantially does not expand or contract upon twisting of the stent.

10. The stent of claim 7 wherein the stent comprises at least a helical portion of the stent that comprises the diameter of the stent that at least one of controllably expands or controllably contracts upon twisting of the stent.

11. The stent of claim 7 wherein the stent comprises at least one lock configured to maintain the stent at a desired diameter after the stent has been twisted.

12. The stent of claim 7 configured for insertion into a lumen of a vessel of a patient, comprising at least one nesting winding having at least one adjacent strut pair having an integral length ratio of at least about 1:2.

13. The stent of claim 7 wherein the stent is a stent configured for insertion into the lumen of a vessel of a patient, comprising: multiple nesting windings having alternating strut lengths and a strut pair length ratio with an integral length ratio of at least about 1:2.

14. The stent of claim 13 wherein the adjacent strut pairs have a length ratio of 1:2.

15. The stent of claim 7 comprising multiple nesting windings having uniform strut lengths and at least one pair of adjacent struts with a length ratio of about 1:2.

16. The stent of claim 7 wherein the stent is a stent comprising substantially squared ends and substantially uniform architectural properties throughout the stent.

17. The stent of claim 7 wherein the stent is configured to comprise substantially only the nesting windings.

18. The stent of claim 1 constructed of tubing or wire.

19. The stent of claim 2 constructed of tubing or wire.

20. The stent of claim 3 constructed of tubing or wire.

21. The stent of claim 7 constructed of tubing or wire.

22. A method comprising implanting a stent according to claim 1 into a lumen of a vessel of a patient.

23. The method of claim 22 wherein the method further comprises removing the stent during or after the implantation or repositioning the stent within the lumen during the implantation.

24. The method of claim 22 wherein the method further comprises, during implantation, at least partially temporarily retracting the stent back into a delivery catheter.

25. A method comprising implanting a stent according to claim 3 into a lumen of a vessel of a patient.

26. The method of claim 25 wherein the method further comprises removing the stent during or after the implantation or repositioning the stent within the lumen during the implantation.

27. The method of claim 26 wherein the method further comprises, during implantation, at least partially temporarily retracting the stent back into a delivery catheter.

28. A method comprising implanting a stent according to claim 1 into a lumen of a vessel of a patient.

29. The method of claim 6 wherein the method further comprises removing the stent during or after the implantation or repositioning the stent within the lumen during the implantation.

30. The method of claim 28 wherein the method further comprises, during implantation, at least partially temporarily retracting the stent back into a delivery catheter.

31. A minimally invasive surgical tool comprising a distal portion configured for insertion into the lumen of a vessel of a patient, the distal portion comprising multiple nesting windings having alternating strut lengths and a strut pair length ratio with an integral length ratio of at least about 1:2.

32. The tool of claim 24 wherein the distal portion is configured such that a diameter of the distal portion at least one of controllably expands or controllably contracts upon twisting of the distal portion.
